(19) United States
(12) Patent Application Publication
(10) Pub. No.: US 2008/0279968 A1
(43) Pub. Date: Nov. 13, 2008

(54) Composition and method for inducing lipolysis and increasing the metabolism of free fatty acids

(76) Inventors: Marvin HEUER, Mississauga (CA); Ken CLEMENT, Mississauga (CA); Shan CHAUDHURI, Mississauga (CA); Michele MOLINO, Mississauga (CA); Phil APONG, Mississauga (CA)

Correspondence Address: TORYS LLP, 79 WELLINGTON STREET WEST, SUITE 3000, BOX 270, TD CENTRE, TORONTO, ON, M5K 1N2, CA

(21) Appl. No.: 11/747,215

(22) Filed: May 10, 2007

(51) Int. Cl. A61K 36/82; A61P 3/04

(52) U.S. Cl. 424/729

(57) ABSTRACT

A nutritional supplement comprising at least a therapeutically effective amount of green tea extract and a therapeutically effective amount of γ-butyrobetaine is provided by the present invention. The ingredients of the present nutritional supplement act substantially simultaneously to induce lipolysis, resulting in free fatty acid release, and increase the catabolism of free fatty acids via the phosphorylation of perilipins. In an additional aspect of the present invention γ-butyrobetaine ethyl ester is added to the nutritional supplement to provide further synergistic or additional benefits. Both a composition and a method are provided by the present disclosure.

### RELATED APPLICATIONS

This application is related to co-pending U.S., patent application: Ser. No. ______, entitled “Composition for facilitating a more favorable blood lipid profile and increasing the metabolism of free fatty acids via increased nitric oxide levels” filed on May 10, 2007, the contents of which are hereby incorporated by reference in there entirety.

### FIELD OF THE INVENTION

The present invention relates to a nutritional supplement for inducing lipolysis and increasing the catabolism of free fatty acids via the phosphorylation of perilipins. More specifically, the present invention relates to a nutritional supplement comprising a combination of green tea extract and γ-butyrobetaine.

### BACKGROUND OF THE INVENTION

Obesity, a condition of excessive body fat, generally results from more food being consumed than is being used. Stemming from excessive body fat, several health-related concerns have been linked to obesity and being overweight, such as increased morbidity, hypertension, coronary heart disease, type 2 diabetes mellitus, stroke and even some forms of cancer (Curioni C, Andre C, Veras R. Weight reduction for primary prevention of stroke in adults with overweight or obesity. Cochrane Database Syst Rev. Oct. 18, 2006; (4):CD006062). Obesity has become an increasingly widespread and predominant health concern. According to the World Health Organization (WHO) obesity is considered a multifactorial chronic disease which is increasing in frequency (Curioni C, Andre C, Veras R. Weight reduction for primary prevention of stroke in adults with overweight or obesity. Cochrane Database Syst Rev. Oct. 18, 2006;(4):CD006062).

One of the main contributing factors in obesity is overeating, which results in an excess of energy being consumed in relation to the amount of energy being expended by an individual. This excess energy is then commonly stored as fat. A simplified determination of an individual's body weight is essentially governed by the net effect of energy consumed versus energy expended. Daily energy expenditure consists of three components: basal metabolic rate, adaptive thermogenesis and physical activity (Westerterp K R. Diet induced thermogenesis. Nutr Metab (Lond). Aug. 18, 2004 ;1(1):5). All of the aforementioned components must be in a balance with energy expenditure in an individual, that is, energy or food intake being such that an individual does not gain nor lose body weight. Therefore, in order for a person to lose body weight from a reduction in adipose tissue, more energy must be expended by the individual than is taken into the body.

With the unprecedented rise in obesity throughout the world, there exists both a need and want from individuals for improved aids, methods and interventions directed to reducing body fat and maintaining lowered levels of body fat. These needs have led to intensive study of the various mechanisms of fat metabolism. One such mechanism that has shown promise is the arginine-nitric oxide pathway. Nitric oxide (NO), which is synthesized from arginine by all cell types, has been shown to be a key signal molecule involved in adipose tissue biology by influencing adipogenesis and insulin-stimulated glucose uptake.

There are four major mechanisms by which NO has been shown to influence energy metabolism. As a first mechanism, NO has been shown to increase the phosphorylation of both hormone-sensitive lipase and perilipin. Perilipin is a protein which coats lipid droplets in adipocytes and acts to protect triglycerides from hormone-sensitive lipases, which break lipids into glycerol and free fatty acids. When the perilipin is phosphorylated its conformation changes and the lipids stored within are exposed to hormone-sensitive lipase, thus enacting hormone-senstive-mediated lipolysis. Additionally, as a second mechaninsm of energy metabolism, NO has been shown to stimulate the phosphorylation of adenosine-3′,5′-monophosphate activated protein kinases. Activation of these kinases causes a decrease in levels of malonyl-CoA, which plays a key role in the chain elongation of fatty acid biosynthesis by providing 2-carbon units to fatty acids. The fatty acids are then comitted to fatty acid chain synthesis. Additionally, activation of these kinases decreases the expression of genes related to lipogenesis and gluconeogenesis. Thirdly, NO has been shown to increase blood flow to insulin-sensitive tissue, thereby promoting substrate uptake and product removal. Lastly, NO has been shown to activate the expression of peroxisome proliferator-activated receptors leading to enhanced mitochondrial biogenesis and oxidative phosphorylation.

### SUMMARY OF THE INVENTION

The present invention is directed towards a nutritional supplement, comprising at least a therapeutically effective amount of green tea extract, and a therapeutically effective amount of γ-butyrobetaine. The composition of the present invention may further comprise γ-butyrobetaine ethyl ester or similar additional derivatives of γ-butyrobetaine. The ingredients of the present nutritional supplement act substantially simultaneously to induce lipolysis, resulting in free fatty acid release, and to increase the catabolism of free fatty acids via the phosphorylation of perilipins. Both a composition and a method are provided by the present disclosure.

### DETAILED DESCRIPTION OF THE INVENTION

In the following description, for the purposes of explanations, numerous specific details are set forth in order to provide a thorough understanding of the present invention. It will be apparent, however, to one of ordinary skill in the art that the present invention may be practiced without these specific details.

The present invention is directed towards a nutritional supplement, for inducing lipolysis and increasing the catabolism of the resultant free fatty acids via the phosphorylation of perilipins. The composition of the present invention comprises at least a combination of green tea extract and γ-butyrobetaine.

The term ‘γ-butyrobetaine’ as used herein is understood to represent gamma-butyrobetaine, also known as, butyrobetaine, deoxycarnitine, actinine, 4-butyrobetaine, or 4-trimethylamniobutyrate. Additionally, as used herein, ‘γ-butyrobetaine’ also includes derivatives of gamma-butyrobetaine wherein said derivatives are esterified forms of γ-butyrobetaine, amides of γ-butyrobetaine, and salts of γ-butyrobetaine, as well as other derivatives of γ-butyrobetaine particularly those derivatives having similar pharmacoproperties to γ-butyrobetaine upon metabolism to an active form as defined by the scope of the disclosure.

A used herein, the term ‘nutritional composition’ includes dietary supplements, diet supplements, nutritional supplements, supplemental dietary and other compositions similarly envisioned and termed not belonging to the conventional definition of pharmaceutical interventions as is known in the art. Furthermore, ‘nutritional compositions’ as disclosed herein belong to category of compositions having at least one physiological function when administered to a mammal by conventional routes of administration.

Green Tea Extract (ECGC, Catechins and Polyphenols)

The active compounds of green tea are a family of polyphenols wherein tannins are the largest of the subgroups the polyphenols contained therein. The most active specific compound of the polyphenols is epigallocatechin gallate (ECGC) which comprises 10-50% of the total catechins found in green tea. Furthermore, caffeine is also a major active component of green tea; however the percentage of caffeine contained in extracts of green tea fluctuates significantly owing to several different factors such as processing, for example.

Green tea principally acts in a beneficial way through the polyphenols' antioxidant activities as evidenced by several laboratory studies. One clinical study has shown that the ingestion of an extract of green tea results in a rapid increase in plasma antioxidant activity (Benzie I F, Szeto Y T, Strain J J, Tomlinson B. Consumption of green tea causes rapid increase in plasma antioxidant power in humans. Nutr Cancer, 1999. 34(1):83-7).

Moreover, green tea has also been shown to be effective in aiding weight loss. This effect may be due to two activities. Green tea both reduces fat digestion and increases energy expenditure (Berube-Parent S, Pelletier C, Dore J, Tremblay A. Effects of encapsulated green tea and Guarana extracts containing a mixture of epigallocatechin-3-gallate and caffeine on 24 h energy expenditure and fat oxidation in men. Br J Nutr, 2005;94(3):432-6). Fat stores provide the energy necessary for the increase in energy expenditure via the oxidation of fat, consequently leading to thermogenesis. In this regard, the β-oxidation of fats resulting from administration of green tea extracts is a result of activities at adenosine receptors. The activation of adenosine receptors on a cell's surface causes an increase in cyclic adenosine monophosphate (cAMP), leading to lipolysis via an increase in epinephrine and norepinephrine.

Additionally, the mechanism of action of green tea may also be, due to an increase in norepinephrine. Catechins, found in green tea, are known to inhibit catechol-O-methyl-transferase (COMT), an enzyme which degrades norepinephrine. In turn, norepinephrine inhibits the degradation as well as increases the production of cyclic adenosine monophosphate (cAMP). Furthermore, increasing norepinephrine levels by the inhibition of norepinephrine uptake results in increased weight loss in both lean and obese mice as evidenced in animal studies (Billes S K, Cowley M A. Inhibition of Dopamine and Norepinephrine Reuptake Produces Additive Effects on Energy Balance in Lean and Obese Mice. Neuropsychopharmacology. April 2007;32(4):822-34). Stemming from increased norepinephrine levels is the result of an increased presence of cAMP which leads to greater activation of protein kinase A, which subsequently activates lipases found in adipose tissue.

It is herein understood by the inventors that increasing the amount of cAMP present in cells will lead to increased protein kinase A activity, resulting in increased activity of lipases and thus greater fatty acid release.

An embodiment of the present invention comprises Green Tea extract. A serving of the nutritional supplement contains from about 0.05 g to about 1.00 g of Green Tea extract. In a preferred embodiment, the Green Tea extract is standardized to about 45% EGCG, about 75% catechins, and about 90% polyphenols.

γ-butyrobetaine (GBB) and Derivatives

γ-butyrobetaine is an intermediate in carnitine biosynthesis in mammals. It is synthesized, from trimethyl lysine, in almost all cell types and then excreted into the blood to be reabsorbed by the kidney and liver. After reabsorption, GBB is converted to carnitine by γ-butyrobetaine dioxygenase. This conversion to carnitine is extremely efficient, thus presence of γ-butyrobetaine in urine is very small (Vaz F M, Wanders R J A. Carnitine biosynthesis in mammals. Biochem J. 2002;361:417-429).

Since γ-butyrobetaine is a precursor to carnitine, its administration has been studied as a way to increase carnitine levels in the body. Carnitine acts as a carrier molecule for fatty acids across the inner mitochondrial membrane. In order for free fatty acids to be catabolized, they must first enter the mitochondria of a cell such that β-oxidation may take place to produce energy. Fatty acids are first activated with the addition of coenzyme A (CoA), then bound to carnitine and transferred across the mitochondrial inner membrane. After transport across the mitochondrial membrane, the carnitine is removed and the fatty acid is oxidized to produce energy.

Additionally, the administration of γ-butyrobetaine to rats (Sjakste N, Kleschyov J L, Baumane L, Dzintare M, Meirena D, Sjakste J, Sydow K, Munzel T, Kalvinsh I. Endothelium- and nitric oxide-dependent vasorelaxing activities of gamma-butyrobetaine esters: possible link to the antiischemic activities of mildronate. Eur J Pharmacol. Jul. 8, 2004; 495(1):67-73 (Abstract)), provides vasodilating activities. These vasodilating activities were attributed to increases in nitric oxide concentrations in blood. Since nitric oxide has been shown to increase energy metabolism, it is herein understood by the inventors that increases in NO levels will lead to increased catabolism of fatty acids being released from adipocytes, thereby reducing the fat content in the body. The mechanisms of which have been previously discussed above.

An embodiment of the present invention comprises γ-butyrobetaine or derivatives thereof. A serving of the nutritional supplement contains from about 0.005 g to about 0.050 g of γ-butyrobetaine or derivatives thereof.

Since fatty acids which have been release can be reabsorbed and re-stored again as fat, it is desirable to eliminate the fatty acids which have been released before they are reabsorbed. As such, the present invention aims to not only release fatty acids stored in the body, but to substantially simultaneously oxidized them to energy which can be emitted from body of an individual as heat. The oxidized fatty acids cannot be reabsorbed and re-stored as fat, thus weight loss results.

In an embodiment of the present invention, which is set forth in greater detail in Example 1, the nutritional supplement comprises an extract of green tea, γ-butyrobetaine, and γ-butyrobetaine ethyl ester. The nutritional supplement is provided in any acceptable and suitable oral dosage form as known in the art. Lipolysis via cAMP signaling and the metabolism of free fatty acids via increased nitric oxide levels and β-oxidation is substantially simultaneously induced and carried out in an individual by administration of the composition of the present invention.

The nutritional supplement of the present invention may be administered in a dosage form having controlled release characteristics i.e. time-release. Furthermore, the controlled release may be in forms such as a delayed release of active constituents, gradual release of active constituents, or prolonged release of active constituents. Such active constituents release strategies extend the period of bioavailability or target a specific time window for optimal bioavailability. Advantageously the nutritional supplement may be administered in the form of a multi-compartment capsule which combines both immediate release and time-release characteristics. Individual components of the nutritional supplement may be contained in differential compartments of such a capsule such that specific components are released rapidly while others are time-dependently released. Alternatively, a uniform mix of the various components of the present invention may be divided into both immediate release and time-release compartments to provide a multi-phasic release profile.

While, not wishing to be bound by theory, the present invention is comprised of components which have been shown to inhibit the activity of enzymes which degrade norepinephrine. The resulting increase in the presence of norepinephrine will lead to inhibition of the degradation of cAMP as well as the increased production of cAMP. Increased levels of cAMP present in cells will lead to elevated protein kinase A activity, resulting in increased activity of lipases and thus greater fatty acid release in the body of an individual upon administration of the composition of the present invention.

Additionally, the present invention comprises components which have been shown to lead to, via increased nitric oxide levels, increased phosphorylation of hormone-sensitive lipase and perilipin. It is herein understood by the inventors that increased phosphorylation of perilipin will result in a greater likelihood of lipids being broken down by lipases, leading to increased release of free fatty acids in the body of an individual upon administration of the composition of the present invention.

Furthermore, increased nitric oxide levels will yield greater phosphorylation of adenosine-3′,5′-monophosphate activated protein kinase, which will cause a decrease in levels of malonyl-CoA and decrease the expression of genes related to lipogenesis and gluconeogenesis. It is herein understood by the inventors that decreased malonyl-CoA levels will result in reduced chain elongation of fatty acids and thus a decrease in fatty acid biosynthesis in the body of an individual upon administration of the composition of the present invention.

In addition, the present invention comprises components that have been shown to lead to, via increased nitric oxide levels, elevated expression of peroxisome proliferator-activated receptors. It is herein understood by the inventors that increased expression of peroxisome proliferator-activated receptors will enhance mitochondrial biogenesis and oxidative phosphorylation, resulting in elevated metabolism of free fatty acids in the body of an individual upon administration of the composition of the present invention.

Further to the aforementioned functions, the present invention comprises components that have been shown to lead to, via increased nitric oxide levels, elevated blood flow to insulin-sensitive tissue. It is herein understood by the inventors that increased blood flow will promote substrate uptake and product removal, thus increasing the movement of free fatty acids throughout the body of an individual of the composition of the present invention.

Additional embodiments of the present invention may also include portions of the composition as fine-milled ingredients. U.S. Non-Provisional patent application Ser. No. 11/709,526 entitled “Method for Increasing the Rate and Consistency of Bioavailability of Supplemental Dietary Ingredients” filed Feb. 21, 2007, which is herein fully incorporated by reference, discloses a method of increasing the rate of bioavailability following oral administration of components comprising supplemental dietary compositions by the process of particle-milling.

Furthermore, additional embodiments of the present invention may be incorporated into specific controlled-release solid dosage forms. U.S. Non-Provisional patent application Ser. No. 11/709,525 entitled “Method for a Supplemental Dietary Composition Having a Multi-Phase Dissolution Profile” filed Feb. 21, 2007, which is herein fully incorporated by reference, discloses a method of achieving a solid oral dosage form with multiple dissolution characteristics for the release of active ingredients.

According to various embodiments of the present invention, the nutritional supplement may be consumed in any form. For instance, the dosage form of the nutritional supplement may be provided as, e.g., a powder beverage mix, a liquid beverage, a ready-to-eat bar or drink product, a capsule, a liquid capsule, a tablet, a caplet, or as a dietary gel. The preferred dosage forms of the present invention are as a caplet or as a liquid capsule.

Furthermore, the dosage form of the nutritional supplement may be provided in accordance with customary processing techniques for herbal and nutritional supplements in any of the forms mentioned above. Additionally, the nutritional supplement set forth in the example embodiment herein disclosed may contain any appropriate number and type of excipients, as is well known in the art. By way of ingestion of the composition of the present invention, a method for substantially simultaneously inducing lipolysis and increasing the catabolism of free fatty acids via the phosphorylation of perilipins. The method of the present invention comprises at least the step of administering to an individual a therapeutically acceptable amount of the composition of the present invention.

Although the following example illustrates the practice of the present invention in one of its embodiments, the example should not be construed as limiting the scope of the invention. Other embodiments will be apparent to one of skill in the art from consideration of the specifications and example.

### EXAMPLE

A nutritional supplement comprising the following ingredients per serving is prepared for consumption as a caplet to be administered before meals:

About 0.46 g of green tea extract which is standardized for 90% polyphenols, 75% catechins, 45% epigallocatechin gallate, about 0.01 g of γ-butyrobetaine (GBB), and about 0.2 g of γ-butyrobetaine ethyl ester.

Extensions and Alternatives

In the foregoing specification, the invention has been described with a specific embodiment thereof; however, it will be evident that various modifications and changes may be made thereto without departing from the broader spirit and scope of the invention.

What is claimed:

1. A method comprising the step of administering to a mammal a composition comprising an effective amount of green tea extract, and an effective amount of γ-butyrobetaine;
wherein the ingredients act substantially simultaneously to induce lipolysis, resulting in free fatty acid release, and increase the catabolism of free fatty acids via the phosphorylation of perilipins.

2. The method of claim 1 wherein malonyl-CoA is decreased, thereby reducing fatty acid biosynthesis.

3. The method of claim 1 the expression of peroxisome proliferators-activated receptors, thereby increasing fatty acid metabolism by enhancing mitochondrial biogenesis and oxidative phosphorylation.

4. The method of claim 1 wherein blood flow to insulin-sensitive tissue is enhanced, thereby promoting movement of free fatty acids throughout the body.

5. A composition comprising an effective amount of green tea extract, and an effective amount of γ-butyrobetaine;
wherein the ingredients act substantially simultaneously to induce lipolysis, resulting in free fatty acid release, and increase the catabolism of free fatty acids via the phosphorylation of perilipins.

6. The composition of claim 6 wherein the presence of malonyl-CoA is decreased, thereby reducing fatty acid biosynthesis.

7. The composition of claim 5 wherein expression of peroxisome proliferators-activated receptors is increased, thereby increasing fatty acid metabolism by enhancing mitochondrial biogenesis and oxidative phosphorylation.

8. The composition of claim 5 wherein blood flow to insulin-sensitive tissue is enhanced, thereby promoting movement of free fatty acids throughout the body.

9. A composition comprising from about 0.050 g to about 1.000 g of green tea extract, and from about 0.005 g to about 0.050 g of γ-butyrobetaine; wherein the green tea extract and the γ-butyrobetaine act substantially simultaneously to induce lipolysis, resulting in free fatty acid release, and increase the catabolism of free fatty acids via the phosphorylation of perilipins.

10. The composition of claim 9 wherein the amount of green tea extract is about 0.300 g and the amount of γ-butyrobetaine is about 0.010 g.

11. The composition of claim 9 wherein at least a portion of one or more ingredients is fine-milled.

12. The composition of claim 9 wherein the green tea extract and γ-butyrobetaine are part of a solid oral dosage form having a multi-phasic rate of dissolution.

13. The composition of claim 12 wherein said multi-phasic rate of dissolution comprises a first-phase and a second-phase; whereby said first-phase has a first rate of dissolution said second-phase has a second rate of dissolution.

14. The composition of claim 13, further comprising a third-phase, whereby said third-phase has a third rate of dissolution.
